(19) United States
(12) Patent Application Publication
(10) Pub. No.: US 2009/0281083 A1
(43) Pub. Date: Nov. 12, 2009

(54) Combination Product

(75) Inventors: Anders Ljunggren, Molndal (SE); Peter Morsing, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/911,726

(22) PCT Filed: Apr. 13, 2006
(86) PCT No.: PCT/SE06/00442

§ 371 Date: Oct. 17, 2007

(30) Foreign Application Priority Data
Apr. 18, 2005 (SE) 0500871-9

(51) Int. Cl. A61K 31/519; A61P 9/12; A61P 9/00; A61K 31/55

(52) U.S. Cl. 514/212.07; 514/249

(57) ABSTRACT

There is provided a combination product comprising: (A) BH₄, a functional derivative or biosynthetic precursor thereof, or a pharmaceutically-acceptable derivative of BH₄ or its derivative or precursor; and (B) an inhibitor of the biosynthesis and/or vasopressor function of AII, or a pharmaceutically-acceptable derivative thereof, wherein each of components (A) and (B) is formulated in admixture with a pharmaceutically-acceptable adjuvant, diluent or carrier, as well as the use of such a combination product in the treatment of hypertension and/or conditions characterised by vascular dysfunction and/or lesions.

### FIELD OF THE INVENTION

This invention relates to a new combination of pharmaceutically-active compounds.

### BACKGROUND AND PRIOR ART

The renin-angiotensin system plays a major role in the maintenance and control of blood pressure. In this system, important steps are:
(a) the release of the protease renin, which converts the alpha-2 globulin angiotensinogen to angiotensin I (AI);
(b) the action of angiotensin converting enzyme (ACE) on AI to produce the octapeptide hormone angiotensin II (AII);
(c) the downstream actions of AII, mediated by specific receptors, resulting in vasoconstriction.

The activation by AII of type-1 (AT₁) receptors produces a number of effects that lead to vasoconstriction. For example, binding of AII to AT₁ receptors is know to elicit release of reactive oxygen species (ROSs). The increased oxidative stress induced by release of ROSs then decreases levels of nitric oxide, a powerful vasodilator, which is catabolised to species such as peroxynitrite (see Hypertension 37, 1047-1052 (2001) and ibid. 45, 1-6 (2005).

Thus, because they either inhibit the biosynthesis or function of AII, inhibitors of renin or ACE, as well as antagonists of AII receptors, have found utility in the treatment of hypertension.

For example, compounds such as candesartan (an AII receptor antagonist having selectivity for AT₁ receptors) have been demonstrated to be particularly effective antihypertensive agents (see Blood Pressure 11, 293 (2002)).

The reduction in blood pressure achieved by the administration of agents such as ACE inhibitors or AT₁ receptor antagonists is know to be beneficial in the control of various disease states associated with hypertension. For example, it is known that ACE inhibitors and AT₁ receptor antagonists can delay or prevent the development of microvascular diseases such as diabetic nephropathy or diabetic retinopathy (see, for example, WO 02/10182, EP 0 622 077, EP 0 914 158, WO 2004/096211 and J. Renin-Angiotensin-Aldosterone System 2 (suppl. 1), S191-S195 (2001)).

Tetrahydrobiopterin (BH₄) is an essential cofactor for various enzymes involved in the biosynthesis of a number of bioactive molecules. For example, BH₄ is a cofactor for all three isoforms of nitric oxide synthase, as well as for several hydroxylases (phenylalanine hydroxylase, tyrosine-3-hydroxylase and tryptophan-5-hydroxylase).

A diverse range of disorders are know to arise in individuals having BH₄ deficiency, the nature of the disorder depending upon which enzyme(s) in the biosynthetic pathway to BH₄ is defective in those individuals (see The Metabolic and Molecular Bases of Inherited Diseases, pages 1725-76 (“Disorders of tetrahydrobiopterin and related biogenic amines”), Scriver, C. R.; Beaudet, A. L.; Sly, W. S.; Valle, D.; Childs B.; and Vogelstein B., eds., New York, McGraw-Hill (2001)).

Through its role in the biosynthesis of nitric oxide (a key signalling molecule in vascular homeostasis), BH₄ has recently been identified as a potential therapeutic target in the regulation of endothelial nitric oxide synthase function in vascular disease (see: Nephron Physiol. 94, 6 (2003); Nephrol. Dial Transplant. 19, 2223 (2004); and Atheroscler. Thromb. Vasc. Biol. 24, 1 (2004)).

The biosynthesis of BH₄ is known to proceed via the sepiapterin reductase-catalysed reduction of either 6-pyruvoyl tetrahydrobiopterin or sepiapterin. The reduction of the former compound provides BH₄ directly, whereas reduction of sepiapterin provides 7,8-dihydrobiopterin, which is subsequently reduced by dihydrofolate reductase to provide BH₄. The compounds 6-pyruvoyl tetrahydrobiopterin, sepiapterin and 7,8-dihydrobiopterin thus represent biosynthetic precursors of BH₄.

There is no disclosure or suggestion in any of the above-mentioned documents of a combination comprising BH₄, or a functional derivative or biosynthetic precursor thereof, and an agent that inhibits the biosynthesis and/or vasopressor function of AII.

### DISCLOSURE OF THE INVENTION

According to a first aspect of the invention there is provided a combination product comprising:
(A) BH₄, a functional derivative or biosynthetic precursor thereof, or a pharmaceutically-acceptable derivative of BH₄ or its derivative or precursor; and
(B) an inhibitor of the biosynthesis and/or vasopressor function of AII, or a pharmaceutically-acceptable derivative thereof,
   wherein each of components (A) and (B) is formulated in admixture with a pharmaceutically-acceptable adjuvant, diluent or carrier.
   BH₄, a functional derivative or biosynthetic precursor thereof, or a pharmaceutically-acceptable derivative of BH₄ or its derivative or precursor may hereinafter be referred to as “component (A)”.

Similarly, an inhibitor of the biosynthesis and/or vasopressor function of AII, or a pharmaceutically-acceptable derivative thereof may hereinafter be referred to as “component (B)”.

When used herein, the term “functional derivative of BH₄” includes references to molecules based upon the pyrazinopyrimidone ring system (which ring system is optionally in 5,6-dihydro- or 5,6,7,8-tetrahydro-form) that are capable of increasing the production of nitric oxide by one or more isoforms of nitric oxide synthase. Whether a compound has such capability may be determined non-inventively by those skilled in the art by using known techniques, such by electrochemical monitoring of NO production in the presence and absence of a test compound.

Compounds that are functional derivatives of BH₄ are known in the art and, in this respect, include compounds disclosed in EP 0 164 964 A1, EP 0 983 765 A1 and WO 2004/058268 (the disclosures of which documents are hereby incorporated by reference), such as 1′,2′-diacetyl-5,6,7,8-tetrahydrobiopterin, 6-methyl-5,6,7,8-tetrahydrobiopterin, 6-hydroxymethyl-5,6,7,8-tetrahydrobiopterin and 6-phenyl-5,6,7,8-tetrahydrobiopterin.

When used herein, the term “biosynthetic precursor of BH₄” includes references to pterins that are in a higher oxidation state than BH₄ (either through the presence of additional unsaturation in the piperazinopyrimidone ring system or the replacement of one or both hydroxyl groups with oxo on the substituent at the 3-position of that ring system) and that are precursors of BH₄ in the biosynthetic pathway to that molecule. As such, biosynthetic precursors of BH₄ that may be mentioned include 6-pyruvoyl tetrahydrobiopterin, sepiapterin and 7,8-dihydrobiopterin.

In one embodiment of the invention, the term “BH₄, a functional derivative or biosynthetic precursor thereof” includes references to compounds of formula I,

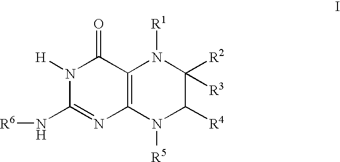

wherein R¹ and R² either independently represent H or C₁₋₂ allyl, or together represent a bond between the C- and N-atoms to which they are attached;

R³ represents aryl or C₁₋₄ alkyl, which alkyl group is optionally substituted by one or more substituents selected from aryl, OR⁷ and oxo;

R⁷ represents, independently at each occurrence, H or C(O)R⁸;

R⁸ represents C₁₋₄ alkyl, aryl, C₁₋₄ alkoxy and C₁₋₄ alkylamino;

R⁴ and R⁵ either independently represent H or C₁₋₂ alkyl, or together represent a bond between the C- and N-atoms to which they are attached; and

R⁶ represents H or C(O)—C₁₋₃₁ alkyl.

The term “aryl”, when used herein, includes C₆₋₁₃ aryl (e.g. C₆₋₁₀) groups. Such groups may be monocyclic, bicyclic or tricylic and, when polycyclic, be either wholly or partly aromatic. In this respect, C₆₋₁₃ aryl groups that may be mentioned include phenyl, naphthyl, 1,2,3,4-tetrahydronaphthyl, indanyl, indenyl, fluorenyl and the like. For the avoidance of doubt, the point of attachment of substituents on aryl groups may be via any carbon atom of the ring system.

Unless otherwise specified, aryl groups may be substituted by one or more substituents selected from —OH, cyano, halo, nitro, C₁₋₆ allyl, C₁₋₆ alkoxy, —N(R^{9a})R^{9b}, —C(O)R^{9c}, C(O)OR^{9d}, —C(O)N(R^{9e})R^{9f}, —N(R^{9g})C(O)R^{9h}, —N(R⁹ⁱ)S(O)₂R^{10a}, —S(O)₂N(R^{9j})(R^{9k}), —S(O)₂R^{10b} and/or —OS(O)₂R^{10c}, (wherein R^{9a} to R^{9k} independently represent H or C₁₋₄ alkyl and R^{10a} to R^{10c} independently represent C₁₋₄ alkyl). When substituted, aryl and aryloxy groups are preferably substituted by between one and three substituents. For the avoidance of doubt, the point of attachment of substituents on aryl groups may be via any carbon atom of the ring system.

The term “halo”, when used herein, includes fluoro, chloro, bromo and iodo.

Unless otherwise specified, alkyl groups and alkoxy groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such allyl and alkoxy groups may also be part cyclic/acyclic. Such alkyl and alkoxy groups may also be saturated or, when there is a sufficient number (i.e. a minimum of two) of carbon atoms, be unsaturated and/or interrupted by one or more oxygen and/or sulfur atoms. Unless otherwise specified, alkyl and alkoxy groups may also be substituted by one or more halo, and especially fluoro, atoms.

Preferred compounds of formula I include those in which:

R¹ and R² either both represent H or together represent a bond between the C- and N-atoms to which they are attached;

R³ represents phenyl (optionally substituted by one to three substituents selected from halo, C₁₋₃ alkyl and C₁₋₃ alkoxy) or C₁₋₄ allyl, which alkyl group is optionally substituted by one or two substituents selected from OR⁷ and oxo;

R⁷ represents, independently at each occurrence, H or C(O)—C₁₋₃ alkyl;

R⁴ represents H;

R⁵ represents H;

R⁶ represents C(O)-[optionally mono- or di-unsaturated C₁₋₃₁ n-alkyl] or, particularly, H.

More preferred compounds of formula I include those in which R³ represents:

(a) —CH₃;

(b) —CH₂OH;

(c) —C(O)C(O)CH₃;

(d) —C(O)CH(OH)CH₃;

(e) —CH(OH)CH(OH)CH₃;

(f) —CH(OC(O)CH₃)CH(OC(O)CH₃)CH₃; or

(g) unsubstituted phenyl.

When R⁶ represents C(O)-[optionally mono- or di-unsaturated C₁₋₃₁ n-alkyl], compounds of formula I that may be mentioned include those in which R⁶ represents a decanoyl, palmitoyl, stearoyl or linoleoyl group.

When used herein, the term “an inhibitor of the biosynthesis and/or vasopressor function of AII” includes references to renin inhibitors, ACE inhibitors and AT₁ receptor antagonists. As such, inhibitors of the biosynthesis and/or vasopressor function of AII that may be mentioned include:
(i) renin inhibitors such as aliskiren, ditelciren, enalkiren, remikiren, terlakiren, zankiren and the like;
(ii) ACE inhibitors such as benazepril, captopril, cilazapril, enalapril, fosinopril, lisinopril, moexepril, perindopril, quinapril, ramipril, trandolapril and the like; and
(iii) AT₁ receptor antagonists such as candesartan, eprosartan, irbesartan, losartan, olmesartan, telmisartan, tasosartan, valsartan and the like.

Other renin inhibitors that may be mentioned include those disclosed in:

(a) U.S. Pat. No. 5,559,111 and EP 0 678 503 (relevant to aliskiren);
(b) EP 0 173 481 (relevant to ditekiren);
(c) EP 0 311 012 (relevant to enallciren);
(d) EP 0 416 373 (relevant to remikiren);
(e) EP 0 266 950 (relevant to terlakiren); and
(f) EP 0 456 185 (relevant to zankiren),
the disclosures of which documents are hereby incorporated by reference.

Other ACE inhibitors that may be mentioned include those disclosed in US patent numbers:

(a) U.S. Pat. Nos. 4,572,909, 4,879,303 and 6,162,802 (relevant to benazepril);
(b) U.S. Pat. No. 5,238,924 (relevant to captopril);
(c) U.S. Pat. Nos. 4,703,038 and 4,803,081 (relevant to enalapril);
(d) U.S. Pat. Nos. 4,337,201 and 4,384,123 (relevant to fosinopril);
(e) U.S. Pat. Nos. 4,508,729 and 5,162,362 (relevant to perindopril);
(f) U.S. Pat. Nos. 4,743,450, 5,684,016 and 5,747,504 (relevant to quiapril);
(g) U.S. Pat. Nos. 4,587,258, 5,061,722 and 5,403,856 (relevant to ramipril); and
(h) U.S. Pat. Nos. 4,933,361, 5,744,496 and 5,721,244 (relevant to trandolapril),
the disclosures of which documents are hereby incorporated by reference.

Other AT₁ receptor antagonists that may be mentioned include those disclosed in US patent numbers:

(a) U.S. Pat. Nos. 5,196,444, 5,534,534, 5,703,110 and 5,705,517 (relevant to candesartan);
(b) U.S. Pat. Nos. 5,185,351 and 5,656,650 (relevant to eprosartan);
(c) U.S. Pat. Nos. 5,270,317, 5,994,348 and 6,342,247 (relevant to irbesartan);
(d) U.S. Pat. Nos. 5,138,069, 5,153,197, 5,608,075 and 5,210,079 (relevant to losartan);
(e) U.S. Pat. No. 5,616,599 (relevant to olmesartan);
(f) U.S. Pat. Nos. 5,591,762, 5,864,043 and 6,358,986 (relevant to telmisartan); and
(g) U.S. Pat. Nos. 5,399,578 and 6,294,197 (relevant to valsartan),
the disclosures of which documents are hereby incorporated by reference.

In one embodiment of the invention, the inhibitor of the biosynthesis and/or vasopressor function of AII is one or more of the ACE inhibitors and, particularly, the AT₁ receptor antagonists mentioned above.

In another embodiment of the invention, the inhibitor of the biosynthesis and/or vasopressor function of AII is one or more of benazepril, captopril, cilazapril, enalapril, fosinopril, lisinopril, moexepril, perindopril, quinapril, ramipril, trandolapril, candesartan, eprosartan, irbesartan, losartan, olmesartan, telmisartan, tasosartan and valsartan.

In a further embodiment of the invention, the inhibitor of the biosynthesis and/or vasopressor function of AII is eprosartan, irbesartan, losartan, olmesartan, telmisartan, tasosartan, valsartan or, particularly, candesartan (optionally in prodrug form, such as candesartan cilexetil).

The combination product according to the invention provides for the administration of component (A) in conjunction with component (B), and may thus be presented either as separate formulations, wherein at least one of those formulations comprises component (A) and at least one comprises component (B), or may be presented (i.e. formulated) as a combined preparation (i.e. presented as a single formulation including component (A) and component (B)).

Thus, there is further provided:
(1) a pharmaceutical formulation including BH₄, a functional derivative or biosynthetic precursor thereof, or a pharmaceutically-acceptable derivative of BH₄ or its derivative or precursor, and an inhibitor of the biosynthesis and/or vasopressor function of AII, or a pharmaceutically-acceptable derivative thereof, in admixture with a pharmaceutically-acceptable adjuvant, diluent or carrier (which formulation is hereinafter referred to as a “combined preparation”); and
(2) a kit of parts comprising components:
   (I) a pharmaceutical formulation including BH₄, a functional derivative or biosynthetic precursor thereof, or a pharmaceutically-acceptable derivative of BH₄ or its derivative or precursor, in admixture with a pharmaceutically-acceptable adjuvant, diluent or carrier; and
   (II) a pharmaceutical formulation including an inhibitor of the biosynthesis and/or vasopressor function of AII, or a pharmaceutically-acceptable derivative thereof, in admixture with a pharmaceutically-acceptable adjuvant, diluent or carrier, which components (I) and (II) are each provided in a form that is suitable for administration in conjunction with the other.

Component (I) of the kit of parts is thus component (A) in admixture with a pharmaceutically-acceptable adjuvant, diluent or carrier. Similarly, component (II) is component (B) in admixture with a pharmaceutically-acceptable adjuvant, diluent or carrier.

According to a further aspect of the invention, there is provided a method of making a kit of parts as defined above, which method comprises bringing a component (I), as defined above, into association with a component (II), as defined above, thus rendering the two components suitable for administration in conjunction with each other.

By bringing the two components “into association with” each other, we include that components (I) and (II) of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, there is further provided a kit of parts comprising:
(1) one of components (I) and (II) as defined herein; together with
(2) instructions to use that component in conjunction with the other of the two components.

The kits of parts described herein may comprise more than one formulation including an appropriate quantity/dose of component (A), and/or more than one formulation including an appropriate quantity/dose of component (B), in order to provide for repeat dosing. If more than one formulation (comprising either active compound) is present, such formulations may be the same, or may be different in terms of the dose of component (A) or component (B), chemical composition and/or physical form.

The combination products according to the invention find utility in the treatment of hypertension and conditions characterised by vascular (e.g. microvascular) dysfunction and/or lesions.

When used herein, the term “conditions characterised by (micro)vascular dysfunction and/or lesions” include conditions in which oxidative stress in the endothelial tissues results in constriction and/or scarring of (micro)vasculature. Conditions that may be mentioned in this respect include left ventricular dysfunction, heart failure, myocardial infarction, angina pectoris, coronary artery disease, peripheral artery disease, atherosclerosis, Reynaud's disease, migraine, cerebral apoplexy, retinopathy, neuropathy and, particularly, nephropathy, glomerulonephritis and glomerulosclerosis. Specific conditions that may also be mentioned also include diabetic retinopathy, diabetic neuropathy and, particularly, diabetic nephropathy, diabetic glomerulonephritis and diabetic glomerulosclerosis.

Thus, a further aspect of the invention provides a method of treatment of hypertension and conditions characterised by microvascular dysfunction and/or lesions which treatment comprises administration of a pharmaceutical formulation including component (A) and component (B), in admixture with a pharmaceutically-acceptable adjuvant, diluent or carrier.

A further aspect of the invention provides a method of treatment of hypertension and conditions characterised by microvascular dysfunction and/or lesions, which comprises administration of:
(a) a pharmaceutical formulation including component (A) in admixture with a pharmaceutically-acceptable adjuvant, diluent or carrier; in conjunction with
(b) a pharmaceutical formulation including component (B) in admixture with a pharmaceutically-acceptable adjuvant, diluent or carrier,
   to a patient suffering from, or susceptible to, such a condition.

For the avoidance of doubt, as used herein, the term “treatment” includes therapeutic and/or prophylactic treatment of a condition.

With respect to the kits of parts as described herein, by “administration in conjunction with”, we include that respective formulations comprising component (A) and component (B) are administered, sequentially, separately and/or simultaneously, over the course of treatment of the relevant condition, which condition may be acute or chronic.

Thus, in respect of the combination product according to the invention, the term “administration in conjunction with” includes that the two components of the combination product (component (A) and component (B)) are administered (optionally repeatedly), either together, or sufficiently closely in time, to enable a beneficial effect for the patient, that is greater, over the course of the treatment of the relevant condition, than if either a formulation comprising component (A), or a formulation comprising component (B), are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term “in conjunction with” includes that one or other of the two formulations may be administered (optionally repeatedly) prior to, after, and/or at the same time as, administration with the other component. When used in this context, the terms “administered simultaneously” and “administered at the same time as” include that individual doses of component (A) and component (B) are administered within 48 hours (e.g. 24 hours) of each other.

When used herein, the term “phammaceutically-acceptable derivative” includes references to salts (e.g. pharmaceutically-acceptable, non-toxic organic or inorganic acid addition salts) and solvates. It will be appreciated by those skilled in the art that the term further includes references derivatives that have, or provide for, the same biological function and/or activity. Moreover, for the purposes of this invention, the term also includes prodrugs (of BH₄, a functional derivative or biosynthetic precursor thereof, and/or of an inhibitor of the biosynthesis and/or vasopressor function of AII). “Prodrugs” of BH₄, or functional derivative or biosynthetic precursor thereof include any composition of matter that, following oral or parenteral administration, is metabolised in vivo to form BH₄, or a functional derivative or biosynthetic precursor thereof, in an experimentally-detectable amount, and within a predetermined time (e.g. within a dosing interval of between 6 and 24 hours (i.e. once to four times daily)). Similarly, “prodrugs” of inhibitors of the biosynthesis and/or vasopressor function of AII include any composition of matter that, following oral or parenteral administration, is metabolised in vivo to an inhibitor of the biosynthesis and/or vasopressor function of AII, in an experimentally-detectable amount, and within a predetermined time (e.g. within a dosing interval of between 6 and 24 hours (i.e. once to four times daily)).

For the avoidance of doubt, the term “parenteral” administration includes all forms of administration other than oral administration.

When the inhibitor of the biosynthesis and/or vasopressor function of AII is an AT₁ receptor antagonist selected from the group consisting of candesartan, eprosartan, olmesartan and valsartan, prodrugs that may be mentioned include esters (e.g. C₁₋₆ alkyl esters, the alkyl group of which is optionally substituted by one or more substituents selected from halo, hydroxy, C₁₋₆ alkoxy, C₃₋₆ cycloalkoxy, C₁₋₆ alkylcarbonyloxy and C₃₋₆ cycloalkylcarbonyloxy, C₁₋₆ alkoxycarbonyloxy and C₃₋₆ cycloalkoxycarbonyloxy) of the free carboxylic acid moieties of those molecules. Preferred prodrugs of the inhibitor of the biosynthesis and/or vasopressor function of AII therefore include candesartan cilexetil (i.e. the 1-(cyclohexyloxycarbonyloxy)ethyl ester of candesartan).

In accordance with the invention, components (A) and (B), may be administered orally, intravenously, subcutaneously, buccally, rectally, dermally, nasally, tracheally, bronchially, topically, by any other parenteral route, or via inhalation, in the form of a pharmaceutical preparation comprising component (A) and/or component (B) in a pharmaceutically-acceptable dosage form. Depending on the disorder, and the patient, to be treated, as well as the route of administration, the compositions may be administered at varying doses.

Preferred modes of delivery are systemic, particularly oral.

In the therapeutic treatment of mammals, and especially humans, component (A) and component (B) will be administered as pharmaceutical formulations in admixture with pharmaceutically-acceptable adjuvants, diluents or carriers, which may be selected with due regard to the intended route of administration and standard pharmaceutical practice.

Suitable formulations for use in administering component (A) are disclosed in the literature, for example as described in inter alia EP 0 164 964, EP 0 983 765 and WO 2004/05826, the disclosures in which documents are hereby incorporated by reference.

Similarly, suitable formulations for use in administering component (B) are disclosed in the literature, for example as described in inter alia European patent application numbers 0 173 481, 0 266 950, 0 311 012, 0 416 373, 0 456 185, 0 622 077 and 0 678 503 and U.S. Pat. Nos. 4,337,201, 4,384,123, 4,508,729, 4,572,909, 4,587,258, 4,703,038, 4,743,450, 4,803,081, 4,879,303, 4,933,361, 5,061,722, 5,138,069, 5,153,197, 5,162,362, 5,185,351, 5,196,444, 5,210,079, 5,238,924, 5,270,317, 5,399,578, 5,403,856, 5,534,534, U.S. Pat. Nos. 5,559,111, 5,591,762, 5,608,075, 5,616,599, 5,656,650, 5,684,016, 5,703,110, 5,705,517, 5,721,244, 5,721,263, 5,744,496, 5,747,504, 5,864,043, 5,958,961, 5,994,348, 6,162,802, 6,294,197, 6,342,247 and 6,358,986, the disclosures in which documents are hereby incorporated by reference. Otherwise, the preparation of suitable formulations, and in particular combined preparations including both component (A) and component (B) may be achieved non-inventively by the skilled person using routine techniques.

The amounts of component (A) and component (B) in the respective formulation(s) will depend on the severity of the condition, and on the patient, to be treated, as well as the compound(s) which is/are employed, but may be determined non-inventively by the skilled person.

Suitable doses of component (A) and component (B) in the therapeutic and/or prophylactic treatment of mammalian, especially human, patients may be determined routinely by the medical practitioner or other skilled person, and include the respective doses discussed in the prior art documents relating to both that are mentioned hereinbefore, the relevant disclosures in which documents are hereby incorporated by reference.

For components (A) and (B), suitable doses for therapeutic or prophylactic purposes are in the range 0.001 to 300 mg/kg body weight (e.g. 0.001 to 50 mg/kg body weight) daily.

For example, for component (A) suitable doses are in the range 1 to 15 mg/kg body weight daily.

In the case of component (B), suitable doses of active compound, prodrugs (e.g. candesartan cilexetil) and derivatives thereof in the therapeutic and/or prophylactic treatment of mammalian, especially human, patients include those in the range of 0.001 to 30 mg/kg body weight daily (for example, when component (B) is candesartan cilexetil, in the range of 0.001 to 3 mg/kg body weight daily). This dose may be achieved, for example, by administering from 1 to 1500 mg (e.g. 2 to 160 mg) of component (B) once daily.

In any event, the physician, or the skilled person, will be able to determine the actual dosage which will be most suitable for an individual patient, which is likely to vary with the condition that is to be treated, as well as the age, weight, sex and response of the particular patient to be treated. The above-mentioned dosages are exemplary of the average case; there can, of course, be individual instances where higher or lower dosage ranges are merited, and such are within the scope of this invention.

When separate formulations are administered, the sequence in which the formulations comprising component (A) and component (B) may be administered (i.e. whether, and at what point, sequential, separate and/or simultaneous administration takes place) may be determined by the physician or skilled person. For example, the sequence may depend upon many factors that will be evident to the skilled person, such as whether, at any time during the course or period of treatment, one or other of the formulations cannot be administered to the patient for practical reasons (e.g. the patient is unconscious and thus unable to take an oral formulation comprising either component (A) or component (B)).

The combination product according to the invention may have the advantage that, when administered to patients, it provides unexpectedly superior blood pressure lowering and organ-protective (e.g. cardio-, cerebro-, retino- or, particularly, nephro-protective) effects compared to administration of either of components (A) or (B) alone. Further, the combination product according to the invention may be more efficacious than, be less toxic than, have a broader range of activity than, be more potent than, produce fewer side effects than, or that it may have other useful pharmacological properties over, similar methods known in the prior art for the treatment of such conditions.

The invention is illustrated, but in no way limited, by the following examples with reference to the figures, which present data gathered from the biological studies described below and in which:

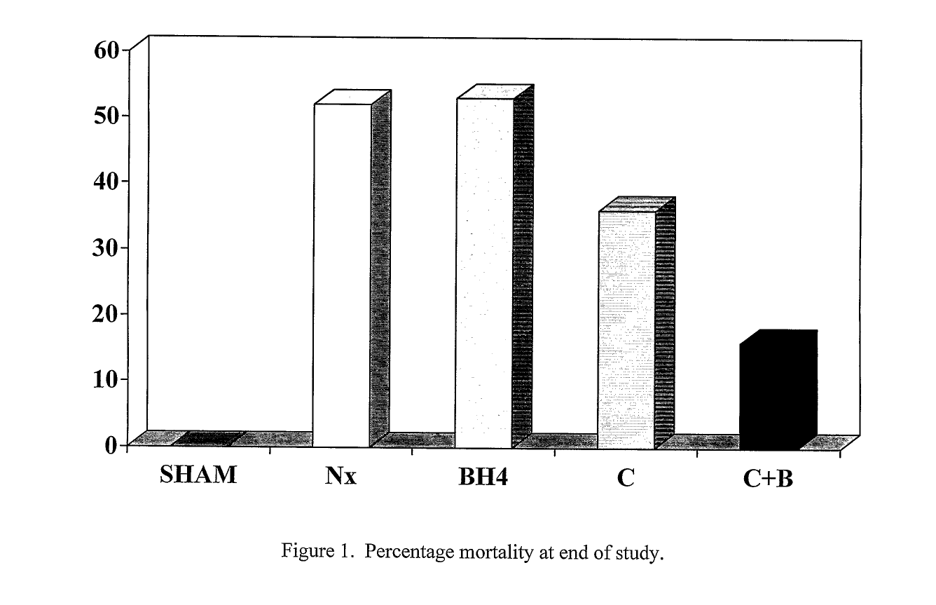

FIG. 1 illustrates percentage mortality at the end of the study period (4 months). Key (FIG. 1):
SHAM—sham-operated rats;
Nx—untreated 5/6 nephrectomised rats;
BH₄—5/6 nephrectomised rats treated with BH₄ (10 mg/kg, i.p.);
C—5/6 nephrectomised rats treated with candesartan cilexetil (5 mg/kg, p.o.);
C+B—5/6 nephrectomised rats treated with candesartan cilexetil (5 mg/kg, p.o.) and BH₄ (10 mg/kg, i.p.).

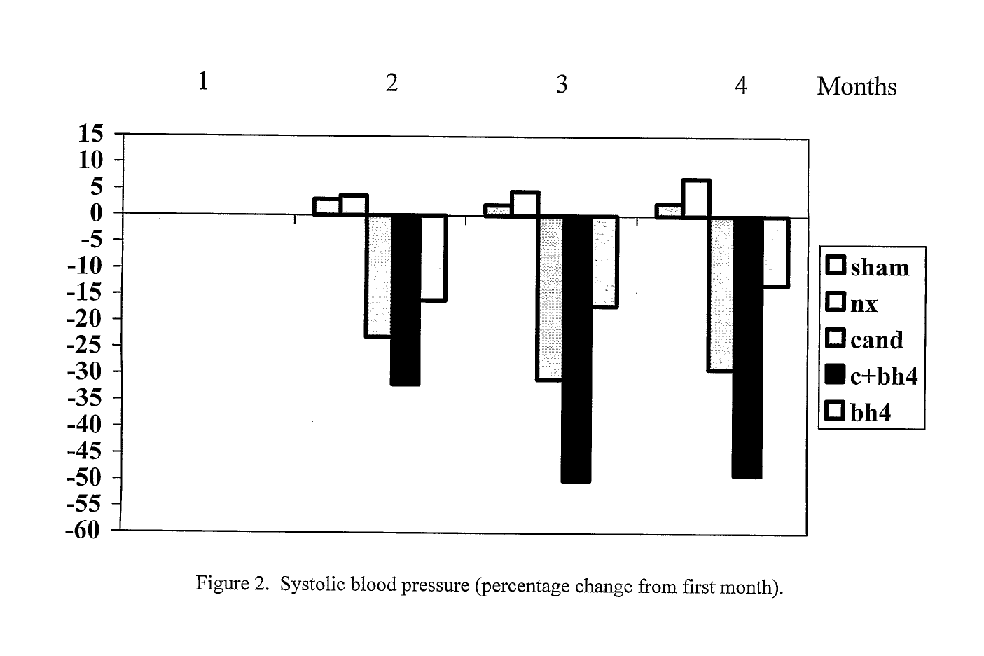

FIG. 2 shows the changes in systolic blood pressure in months 2 to 4 of the study, compared to the pressure measured in month 1 (when no drugs were administered).

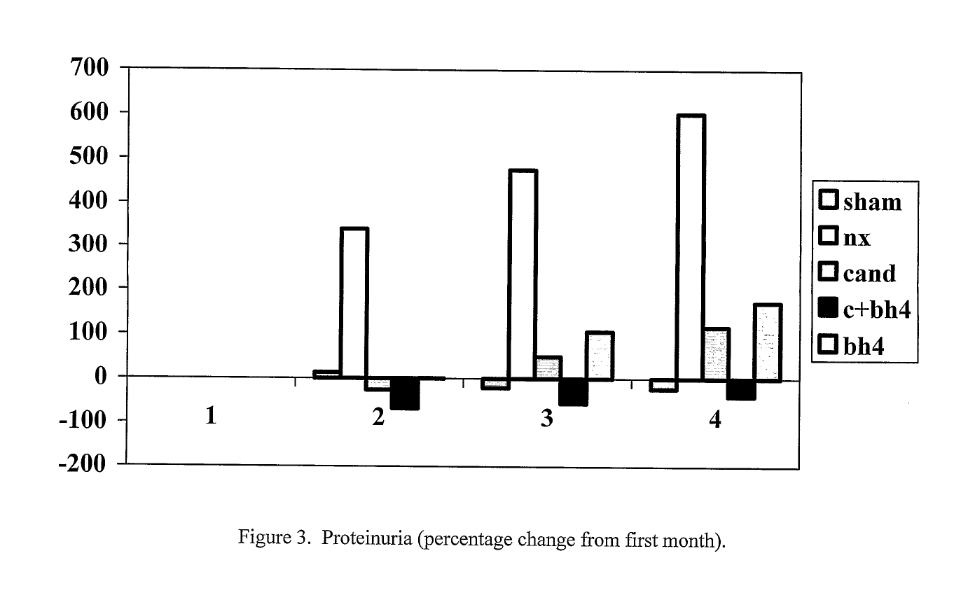

FIG. 3 shows the changes in 24 hour urine protein excretion (mg per mg of creatinine) in months 2 to 4 of the study, compared to the value measured in month 1 (when no drugs were administered).

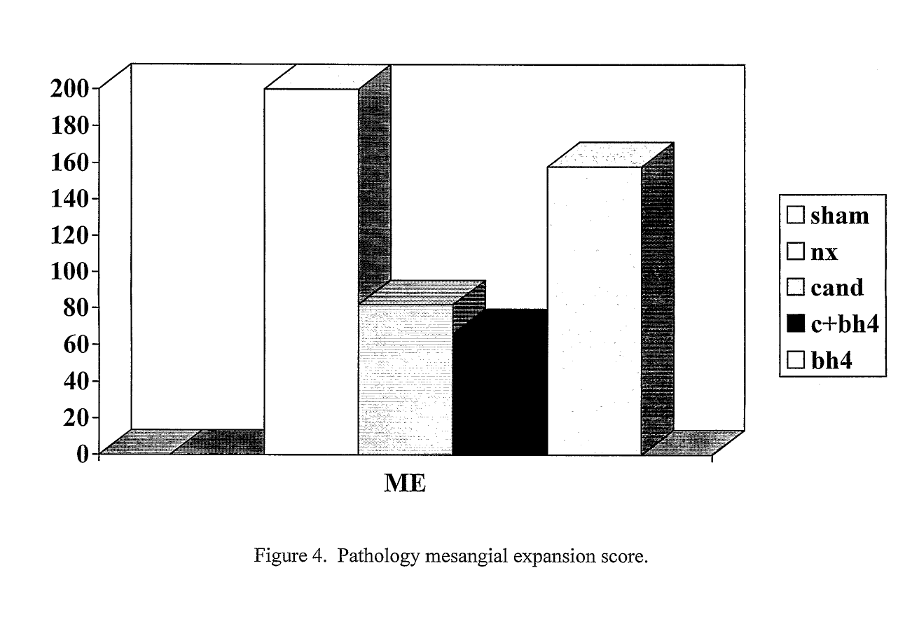

FIG. 4 illustrates the median post-study mesangial expansion scores (determined by histological examination—see below) for the kidneys of the rats from the various groups. Key (FIGS. 2-4):
sham—sham-operated rats;
nx—untreated 5/6 nephrectomised rats;
bh₄—5/6 nephrectomised rats treated with BH₄ (10 mg/kg, i.p.);
cand—5/6 nephrectomised rats treated with candesartan cilexetil (5 mg/kg, p.o.);
c+bh₄—5/6 nephrectomised rats treated with candesartan cilexetil (5 mg/kg, p.o.) and BH₄ (10 mg/kg, i.p.).

### BIOLOGICAL STUDIES

### Protocol

Sixty male Wistar rats, mean weight 324±4 g, were fed a normal chow diet and allowed distilled water ad libitum. The 5/6 nephrectomy was performed according to standard protocol. In brief, the animals were anaesthetized with pentobarbital [35 mg/kg body weight (BW) intraperitoneally] and then underwent a right nephrectomy and ligature of two of the major branches of the left main renal artery (Nx) at day 0. AII the nephrectomies were carried out by one operator. The animals were then randomly assigned to one of five groups, as follows.
Group 1. SHAM (n=12); sham-operated rats.
Group 2. Nx (n=15); untreated 5/6 nephrectomy rats.
Group 3. C (n=11); 5/6 nephrectomy rats to which candesartan cilexetil (AstraZeneca AB) was given daily at a dose of 5 mg/kg BW/day.
Group 4. BH₄ (n=10); 5/6 nephrectomy rats administered BH₄ (Alexis Biochemical, Lausen), 10 mg/kg BW/day intraperitoneally (see Hypertension 38, 1044-1048 (2001), the disclosures of which document are hereby incorporated by reference).
Group 5. C+B (n=12); 5/6 nephrectomy rats to which candesartan cilexetil (AstraZeneca AB) was given daily at a dose of 5 mg/kg BW/day and BH₄ (Alexis Biochemical, Lausen), was administered at a dose of 10 mg/kg BW/day intraperitoneally [5].

Drug administration was begun 30 days after surgery (at a point when the nephrectomised rats have already developed hypertension and renal insufficiency). The study was continued until 50% mortality was observed in the control (Nx) group (which occurred 4 months after surgery). Systolic blood pressure (SBP) was measured in trained unstressed rats 1 day before surgery and at 30, 60, 90 and 120 days, by tail cuff manometry using an automated sphygmomanometer (Narco Bio Systems, Austin, Tex., USA). The Animal Care and Use Committee of Meir Hospital approved all procedures.

### Clinical and Laboratory Evaluations

Serum albumin and cholesterol (as nutritional indices) were assayed at the end of the study. Serum creatinine was determined 1 day before and at 30, 60, 90 and 120 days of the study. Twenty-four hour urine collections were obtained during two consecutive days before surgery and at 30, 60, 90 and 120 days after surgery for the measurement of creatinine clearance and proteinuria. Rats were kept in metabolic cages 3 days before each urine collection and were fed a low nitrate diet containing 0.35 g NaCl, 20 g protein and 1.17 g arginine per 100 g. Gentamicin (6 mg/tube) was added to the urine collection tubes to avoid bacterial growth. Serum creatinine, albumin, cholesterol and urinary proteinuria were assayed by standard methods.

### Histological Examinations

Kidneys were fixed in neutral buffered formalin and embedded in paraffin for light microscopic study. Histological sections were stained with haemotoxylin-eosin, periodic acid-Schiff (PAS) and Masson. The histological sections were coded so that the pathologist was unaware of the source of each sample. A minimum of 40 glomeruli from each kidney was examined. A semi-quantitative score was used to assess the severity of glomerular lesions (see Nephrol. Dial. Transplant. 8, 501-506 (1993), the disclosures of which document are hereby incorporated by reference). Tubulointerstitial and vascular damage were assessed on PAS-stained paraffin sections at a magnification of ×100, Score evaluation was in accordance with Adamczak et al. (see J. Am. Soc. Nephrol. 14, 2833-2842 (2003), the disclosures of which document are hereby incorporated by reference).

### Statistical Analysis

Unless otherwise stated, results are expressed as means±SEM. A one-way analysis of variance with Bonferroni correction was performed in the statistical analysis of data. For paired and unpaired groups, t-tests were used as appropriate. A P-value of <0.05 was considered significant.

### Results

### Clinical Evaluation

Due to abnormalities found by way of post-study autopsies, two rats were excluded from the analysis of the “C+B” group (i.e. Group 5).

At a point 4 months into the study (3 months of drug treatment for rats in groups 3 to 5), survival rates were as follows.

| Group | 5/6 Nephrectomy | Treatment | Survival (% age) | |
|---|---|---|---|---|
| 1 | no | none | 12 of 12 | (100%) |
| | (sham operation) | | | |
| 2 | yes | none | 7 of 15 | (46.67%) |
| 3 | yes | candesartan cilexetil, | 7 of 11 | (63.64%) |
| | | 5 mg/kg | | |
| 4 | yes | BH₄, 10 mg/kg | 5 of 10 | (50%) |
| 5 | yes | candesartan cilexetil, | 8 of 10 | (80%) |
| | | 5 mg/kg | | |
| | | and BH₄, 10 mg/kg | | |

Similarly, at a point 4 months into the study (3 months of drug treatment for rats in groups 3 to 5), mesangial expansion scores were as follows.

| Group | 5/6 Nephrectomy | Treatment | ME Score* |
|---|---|---|---|
| 1 | no (sham operation) | none | 0 |
| 2 | yes | none | 200 |
| 3 | yes | candesartan cilexetil, | 82 |
| | | 5 mg/kg | |
| 4 | yes | BH₄, 10 mg/kg | 158 |
| 5 | yes | candesartan cilexetil, | 66 |
| | | 5 mg/kg | |
| | | and BH₄, 10 mg/kg | |

*median value

1. A combination product comprising:
(A) BH₄, a functional derivative or biosynthetic precursor thereof, or a pharmaceutically-acceptable derivative of BH₄ or its derivative or precursor; and
(B) an inhibitor of the biosynthesis and/or vasopressor function of AII, or a pharmaceutically-acceptable derivative thereof,
wherein each of components (A) and (B) is formulated in admixture with a pharmaceutically-acceptable adjuvant, diluent or carrier.

2. A pharmaceutical formulation comprising:
a combination product comprising:
BH₄, a functional derivative or biosynthetic precursor thereof, or a pharmaceutically-acceptable derivative of BH₄ or its derivative or precursor, and
an inhibitor of the biosynthesis and/or vasopressor function of AII, or a pharmaceutically-acceptable derivative thereof, and
a pharmaceutically-acceptable adjuvant, diluent or carrier.

3. A kit of parts comprising:
(I) a pharmaceutical formulation including BH₄, a functional derivative or biosynthetic precursor thereof, or a pharmaceutically-acceptable derivative of BH₄ or its derivative or precursor, in admixture with a pharmaceutically-acceptable adjuvant, diluent or carrier; and
(II) a pharmaceutical formulation including an inhibitor of the biosynthesis and/or vasopressor function of AII, or a pharmaceutically-acceptable derivative thereof, in admixture with a pharmaceutically-acceptable adjuvant, diluent or carrier,
which components (I) and (II) are each provided in a form that is suitable for administration in conjunction with the other.

4. A kit of parts as claimed in claim 3, wherein components (I) and (II) are suitable for sequential, separate and/or simultaneous use in the treatment of hypertension and/or a condition characterised by microvascular dysfunction and/or lesions.

5. A kit of parts as claimed in claim 4, wherein the condition is hypertension, diabetic retinopathy, diabetic nephropathy, glomerulonephritis and/or glomerulosclerosis.

6. A combination product as claimed in claim 1, wherein the component that is BH₄ or a functional derivative or biosynthetic precursor thereof is a compound of formula I, 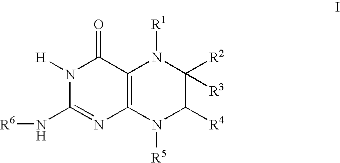
wherein
R¹ and R² either independently represent H or C₁₋₂ alkyl, or together represent a bond between the C- and N-atoms to which they are attached;
R³ represents aryl or C₁₋₄ alkyl, which alkyl group is optionally substituted by one or more substituents selected from aryl, OR⁷ and oxo, and which aryl group is optionally substituted by one or more substituents selected from —OH, cyano, halo, nitro, C₁₋₆ alkyl, C₁₋₆ alkoxy, —N(R^{9a})R^{9b}, —C(O)R^{9c}, —C(O)OR^{9d}, —C(O)N(R^{9e})R^{9f}, —N(R^{9g})C(O)R^{9h}, —N(R⁹ⁱ)S(O)₂R^{10a}, —S(O)₂N(R^{9j})(R^{9k}), —S(O)₂R^{10b}, and —OS(O)₂R^{10c};
R⁷ represents, independently at each occurrence, H or C(O)R⁸;
R⁸ represents C₁₋₄ alkyl, aryl, C₁₋₄ alkoxy or C₁₋₄ alkylamino;
R^{9a} to R^{9k} independently represent H or C₁₋₄ alkyl;
R^{10a} to R^{10c} independently represent C₁₋₄ alkyl;
R⁴ and R⁵ either independently represent H or C₁₋₂ alkyl, or together represent a bond between the C- and N-atoms to which they are attached; and
R⁶ represents H or C(O)—C₁₋₃₁ alkyl.

7. A combination product as claimed in claim 6, wherein R¹ and R² either both represent H or together represent a bond between the C- and N-atoms to which they are attached.

8. A combination product as claimed in claim 6, wherein R³ represents phenyl (optionally substituted by one to three substituents selected from halo, C₁₋₃ alkyl and C₁₋₃ alkoxy) or C₁₋₄ alkyl, which alkyl group is optionally substituted by one or two substituents selected from OR⁷ and; oxo.

9. A combination product as claimed in claim 6, wherein R⁷ represents, independently at each occurrence, H or C(O)—C₁₋₃ alkyl.

10. A combination product as claimed in claim 6, wherein R⁴ and R⁵ both represent H.

11. A combination product as claimed in claim 10, wherein R⁶ represents H.

12. A combination product as claimed in claim 6, wherein R³ represents —CH₃, —CH₂OH, —C(O)C(O)CH₃, —C(O)CH(OH)CH₃, —CH(OH)CH(OH)CH₃, —CH(OC(O)CH₃)CH(OC(O)CH₃)CH₃ or unsubstituted phenyl.

13. A combination product as claimed in claim 1, wherein the inhibitor of the biosynthesis and/or vasopressor function of AII is a renin inhibitor, an ACE inhibitor or an AT₁ receptor antagonist.

14. A combination product as claimed in claim 13, wherein the inhibitor of the biosynthesis and/or vasopressor function of AII is a compound selected from the group consisting of aliskiren, ditekiren, enalkiren, remikiren, terlakiren, zankiren, benazepril, captopril, cilazapril, enalapril, fosinopril, lisinopril, moexepril, perindopril, quinapril, ramipril, trandolapril, candesartan, eprosartan, irbesartan, losartan, olmesartan, telmisartan, tasosartan, valsartan, ciclosidomine, furosemide and saralasin.

15. A combination product as claimed in claim 14, wherein the inhibitor of the biosynthesis and/or vasopressor function of AII is a compound selected from the group consisting of eprosartan, irbesartan, losartan, olmesartan, telmisartan, tasosartan, valsartan or, candesartan, wherein the candesartan is optionally in prodrug form.

16. A method of making a kit of parts as defined in claim 3, which method comprises bringing a component (I) into association with a component (II) thus rendering the two components suitable for administration in conjunction with each other.

17. A kit of parts comprising:
(1) one of components (I) and (II),
(I) a pharmaceutical formulation including BH₄, a functional derivative or biosynthetic precursor thereof, or a pharmaceutically-acceptable derivative of BH₄ or its derivative or precursor, in admixture with a pharmaceutically-acceptable adjuvant, diluent or carrier; or
(II) a pharmaceutical formulation including an inhibitor of the biosynthesis and/or vasopressor function of AII, or a pharmaceutically-acceptable derivative thereof, in admixture with a pharmaceutically-acceptable adjuvant, diluent or carrier;
together with
(2) instructions to use that component in conjunction with the other of the two components.

18-19. (canceled)

20. A method of treatment of hypertension and/or a condition characterised by vascular dysfunction and/or lesions, which comprises administration of a combination product as claimed in claim 1 to a patient suffering from, or susceptible to, such a condition.

21. A method as claimed in claim 20, wherein the condition is hypertension, left ventricular dysfunction, heart failure, myocardial infarction, angina pectoris, coronary artery disease, peripheral artery disease, atherosclerosis, Reynaud's disease, migraine, cerebral apoplexy, retinopathy, neuropathy, nephropathy, glomerulonephritis, glomerulosclerosis, diabetic retinopathy, diabetic neuropathy, diabetic nephropathy, diabetic glomerulonephritis and/or diabetic glomerulosclerosis.

22-24. (canceled)
